(19) United States
(12) Patent Application Publication
(10) Pub. No.: US 2009/0318916 A1
(43) Pub. Date: Dec. 24, 2009

(54) Electrosurgical Methods and Devices Employing Semiconductor Chips

(76) Inventors: Daniel Lischinsky, Ramat Yshay (IL); Yoram Harth, Herzliya (IL)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/548,746

(22) Filed: Aug. 27, 2009

(30) Foreign Application Priority Data
Mar. 3, 2008 (IB) PCT/IB2008/000902

(51) Int. Cl. A61B 18/18

(52) U.S. Cl. 606/33

(57) ABSTRACT

This disclosure relates generally to electrosurgical methods and devices. In one embodiment, an electrosurgical device is provided suitable for applying RF energy to a treatment site. The electrosurgical device comprises one or more RF generators disposed on a semiconductor chip. Also provided are methods of use of such an electrosurgical device, as well as other electrosurgical devices. The methods and devices disclosed herein find utility, for example, in the field of medicine.

### CROSS-REFERENCE TO RELATED APPLICATIONS

This application claims priority to PCT Application Ser. No. PCT/IB2008/000902, filed Mar. 3, 2008, which claims priority to Provisional U.S. Patent Application Ser. No. 60/904,650, filed Mar. 1, 2007. The disclosures of the aforementioned applications are incorporated by reference herein in their entireties.

### TECHNICAL FIELD

This disclosure relates generally to electrosurgical methods and devices. The methods and devices disclosed herein find utility, for example, in the field of medicine.

### BACKGROUND

Radiofrequency (RF) devices are used to ablate or heat different types of tissue. For example, in the field of dermatology RF devices are used to treat aging skin. Skin aging is associated with changes in the upper levels of the skin such as roughness of the skin due to changes in the stratum corneum and epidermis and uneven pigmentation in the epidermis. In the dermis, aging and environmental factors cause the destruction and malfunction of collagen and elastin fibers leading to the formation of wrinkles. Symptoms of skin aging in the epidermis are typically treated by ablative methods such as chemical peels or laser resurfacing. Optical radiation devices such as lasers are used to resurface large areas of the skin. While these lasers are effective in the treatment of the signs of skin aging, resurfacing the whole epidermis is often associated with side effects such as wound infections, prolonged healing times, hyperpigmentation, hypopigmentation, and scarring.

Radiofrequency devices are used to ablate localized skin lesions or to destroy the whole upper surface of the skin. However, whole skin resurfacing methods and devices cause burn-like post treatment reactions associated with prolonged healing times, increased risk of infections, prolonged erythema, scarring, hyperpigmentation, and hypopigmentation.

Symptoms of skin aging in the dermis are typically treated by non-ablative methods, including lasers, intense pulsed light, or RF devices that heat the dermis to trigger renewal of collagen fibers. In order to trigger collagen renewal, some RF devices use bipolar electrodes to increase the heat of dermal skin layers through the creation of electrical currents that flow parallel to the skin surface. These devices use active and return electrodes that are typically positioned relatively close to one another at the treatment site. In some cases, the two electrodes are located on the same probe, and the electrodes alternate between functioning as active and return electrodes. Other RF devices use unipolar or monopolar electrical energy for heating the deep layers of skin. These devices also use an active electrode and a return electrode. The return electrode is typically positioned a relatively large distance from the active electrode (in comparison with bipolar devices). For both unipolar and bipolar devices, current flows along the lowest impedance path between electrodes.

Despite advancements in the use of RF devices for treating biological tissue, there continues to be a need in the art to develop effective electrosurgical devices and methods that are suitable for treating a wide variety of conditions. An ideal electrosurgical method and related devices would be capable of selectively and specifically treating a wide variety of biological tissues and conditions effecting such tissues. Such a method and devices would be simple to use, and would have minimal adverse effects.

### SUMMARY OF THE DISCLOSURE

The present disclosure is directed at addressing one or more of the abovementioned drawbacks of known electrosurgical methods and devices.

In one embodiment, then, the disclosure describes a method for delivering energy to a target site of a patient. The method comprises placing an electrosurgical semiconductor chip into close proximity of the target site and delivering RF energy to the chip.

In another embodiment, the disclosure describes a method for modifying living tissue. The method comprises exposing the tissue to an electric field, wherein the electric field is generated by an electrosurgical device. The electrosurgical device comprises an electrosurgical semiconductor chip. The electrosurgical semiconductor chip comprises an RF generator.

In yet another embodiment, the disclosure describes an electrosurgical system. The electrosurgical system comprises a means for applying RF energy to a target site of a patient. The electrosurgical system further comprises one or more RF generators.

In a still further embodiment, the disclosure describes an electrosurgical system for treating living tissue. The system is configured to deliver RF electrical energy to the living tissue, and comprises a semiconductor chip.

In a further embodiment, an electrosurgical semiconductor chip is described for delivering electrical energy to a treatment site comprising: (a) a semiconductor die comprising one or more RF generators; and (b) a package comprising a plurality of electrical contacts suitable for delivering RF energy to a target site, wherein the electrical contacts are disposed in an array on a treatment surface of the package.

In a still further embodiment, an electrosurgical system is described suitable for treating a target site comprising a plurality of electrodes disposed on a surface of a semiconductor chip package and a means for applying RF energy to at least a portion of the electrodes such that, when RF energy is applied to at least a portion of the electrodes, an electric field suitable for treating the target site is created.

In a still further embodiment, an electrosurgical device is described for applying electrical energy to a target site comprising a semiconductor die, a BGA package, and a means for controllably applying the electrosurgical device to the target site.

In a still further embodiment, a method for applying RF energy to a target site is described, the method comprising placing a semiconductor chip in close proximity to the target site, wherein the semiconductor chip comprises a plurality of electrodes and means for supplying RF energy to at least a portion of the plurality of electrodes.

In a still further embodiment, a method for applying RF energy to a target site is described, the method comprising placing a surface-mount integrated circuit (IC) device in close proximity to the target site and supplying power to the device.

Embodiments of the present disclosure include an electrosurgical semiconductor chip for delivering electrical energy to a treatment site comprising: (a) a semiconductor die comprising one or more RF generators; and (b) a package comprising a plurality of electrical contacts suitable for delivering RF energy to a target site, wherein the electrical contacts are disposed in an array on a treatment surface of the package. One or more RF generators are electrically coupled to at least a portion of the contacts, and the application of RF energy to the contacts creates an electric field suitable for delivering a therapeutic amount of RF energy to the treatment site. The package may be selected from, for example, a BGA (ball grid array), PBGA (plastic BGA), EPBGA (Enhanced plastic BGA), FBGA (Fine BGA), FCBGA (flip-chip BGA), LGA (land-grid array), and PGA (pin grid array). The electrosurgical semiconductor chip may further comprise means for controllably applying the treatment surface to the target site. Such means may include a handle directly or indirectly attached to the package. The electrosurgical semiconductor chip may be disposable and intended for single-use applications, or may be intended for multiple-use applications and/or sterilizable. The RF energy delivered by the device may be sufficient to cause ablation of the tissue. The package may further comprise a second side that is opposite the treatment surface, and comprises means for receiving electrical energy. Such means for receiving electrical energy may comprise a plurality of electrical contacts. The electrical energy may be a DC input signal, and the semiconductor die may further comprise circuitry suitable for converting the DC input signal to RF energy. Such RF energy may be in the form of a plurality of RF signals, and the semiconductor die may further comprise circuitry suitable for independently controlling the phase of each of the plurality of RF signals. Alternatively, the electrical energy may be RF energy, and may be in the form of a plurality of RF signals that are independently phase-controlled.

Embodiments of the present disclosure also include an electrosurgical system suitable for treating a target site comprising a plurality of electrodes disposed on a surface of a semiconductor chip package and a means for applying RF energy to at least a portion of the electrodes such that, when RF energy is applied to at least a portion of the electrodes, an electric field suitable for treating the target site is created. The electrosurgical system may further comprise a semiconductor die. The means for applying RF energy may comprise one or more RF generators, and the one or more RF generators may be disposed on the semiconductor die or separate from the semiconductor die. For example, the means for applying RF energy may comprise a plurality of RF generators, and the electrosurgical system may further comprise means for independently controlling the phase of the output of each of the plurality of RF generators. The plurality of electrodes may be disposed on a treatment surface of the semiconductor chip package, and the semiconductor chip package may further comprise a second surface opposite the treatment surface and comprising electrical contacts that are suitable for receiving an electrical input.

Embodiments of the present disclosure also include an electrosurgical device for applying electrical energy to a target site comprising a semiconductor die, a BGA package, and a means for controllably applying the electrosurgical device to the target site. The means for controllably applying the electrosurgical device may comprise a handle. The BGA package may comprise a substrate and a compound, wherein the handle is attached to the compound. The semiconductor die may comprise an RF generator, and the electrosurgical device may further comprise a power supply (either AC or DC). The power supply may be located within the BGA package or separate from the BGA package. The BGA package may comprise a matrix of contacts disposed on a treatment surface, and may further comprise a plurality of electrical input contacts, wherein the power supply is electrically coupled to at least a portion of the input contacts.

Embodiments of the present disclosure also include a method for applying RF energy to a target site, the method comprising placing a semiconductor chip in close proximity to the target site, wherein the semiconductor chip comprises a plurality of electrodes and means for supplying RF energy to at least a portion of the plurality of electrodes. The semiconductor chip may comprise a BGA package, wherein the plurality of electrodes are ball-type electrical contacts disposed on a surface of the BGA package.

Embodiments of the present disclosure also include a method comprising placing a surface-mount integrated circuit (IC) device in close proximity to the target site and supplying power to the device. The surface-mount IC device may comprise a BGA package and a semiconductor die. Power may be supplied to the device by an external power supply. The surface-mount IC device may further comprise one or more RF generators.

Embodiments of the present disclosure also include a method for delivering RF energy to a treatment site using the electrosurgical semiconductor chip devices of any of the embodiments disclosed herein.

### BRIEF DESCRIPTION OF THE DRAWINGS

FIG. 1 is an example illustration of an electrosurgical chip device as disclosed herein.

FIG. 2 is an example of a circuit diagram for a device according to the disclosure.

FIG. 3 is an example of a block diagram for a device according to the disclosure.

### DETAILED DESCRIPTION OF THE INVENTION

Before describing the present invention in detail, it is to be understood that unless otherwise indicated, this invention is not limited to particular electrosurgical methods, electrosurgical devices, or power sources, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, “a power source” refers not only to a single power source but also to a combination of two or more power sources, “an electrode” refers to a combination of electrodes as well as to a single electrode, and the like.

Unless defined otherwise, all technical and scientific terms used herein have the meaning commonly understood by one of ordinary skill in the art to which the invention pertains. Although any methods and materials similar or equivalent to those described herein may be useful in the practice or testing of the present invention, preferred methods and materials are described below. Specific terminology of particular importance to the description in the present disclosure is defined below.

As used herein, the terms “may,” “optional,” “optionally,” or “may optionally” mean that the subsequently described circumstance may or may not occur, so that the description includes instances where the circumstance occurs and instances where it does not.

As used herein, the term “device” is meant to refer to any and all components of a system. For example, an “electrosurgical device” refers to an electrosurgical system that may comprise components such as electrosurgical probes, electrosurgical semiconductor chips, power sources, connecting cables, and other components.

The terms “treating” and “treatment” as used herein refer to reduction in severity and/or frequency of symptoms, elimination of symptoms and/or underlying cause, prevention of the occurrence of symptoms and/or their underlying cause (e.g., prophylactic therapy), and improvement or remediation of damage.

By “patient,” or “subject” is meant any animal for which treatment is desirable. Patients may be mammals, and typically, as used herein, a patient is a human individual.

The term “phase” as used herein refers to the phase angle of an alternating-current (AC) radiofrequency (RF) voltage (sometimes referred to as an “RF signal” or “RF voltage”). In some cases, the term “phase” also refers to the phase angle difference between two RF voltages. Accordingly, the term “phased RF energy” refers to RF energy that comprises at least two component RF voltages, wherein each component RF voltage independently has a phase.

The terms “electrosurgical chip,” “electrosurgical semiconductor chip,” “semiconductor chip,” or “chip” as used herein refer to any semiconductor chip that is suitable or may be adapted to be suitable for use as an electrosurgical device or as a component of an electrosurgical device. The term “surface-mount integrated circuit device” is used interchangeably with these terms.

Disclosed herein are electrosurgical devices for applying RF energy to a treatment site such as biological tissue. The electrosurgical devices comprise an electrosurgical semiconductor chip that comprises a semiconductor die and a package.

The semiconductor die, as is common understood in the art, comprises various integrated circuits as appropriate. If desired, the circuits can be prepared on a customized chip die with features according to the wishes of the user. The design and preparation of appropriate circuitry for the semiconductor die to achieve the desired functionalities can be accomplished by those of ordinary skill in the art.

For example, the semiconductor die may comprise, where appropriate, one or more RF generators, one or more power supplies, one or more splitter circuits designed to create a plurality of RF output signals from a single RF input signal, and other circuitry as will be appreciated by the skilled artisan.

The package serves to encapsulate (partially or fully) the semiconductor die. In addition, suitable packages for the devices of the invention include those with electrical contacts disposed upon a surface of the package, wherein the surface of the package is suitable to act as a treatment surface. In the methods and devices described herein, the electrical contacts function as electrodes (and the terms “electrodes” and “electrical contacts” are used interchangeably throughout this disclosure), delivering electrical energy to the target site. A typical treatment surface is flat and comprises enough surface area to accommodate a sufficient number of electrical contacts for the intended method of treatment. The electrical contacts are disposed in an array on a treatment surface of the package, and are electrically coupled to one or more RF generators which may be located on the semiconductor die or elsewhere. On a second surface of the package, i.e., one that is opposite the treatment surface, additional electrical contacts may be disposed. Such electrical contacts may be used to deliver electrical power to the electrosurgical chip.

Examples of appropriate packages include the following: BGA (ball grid array), PBGA (plastic BGA), EPBGA (Enhanced plastic BGA), FBGA (Fine BGA), FCBGA (flip-chip BGA), LGA (land-grid array), and PGA (pin grid array) packages. Other packages known in the art, as well as variations and equivalents of such packages, may be used as appropriate in the methods and devices disclosed herein.

The electrosurgical devices may further comprise a power supply that is external to the semiconductor chip, or they may further comprise a power supply that is integrated into the semiconductor chip. The power supply may be alternating current (AC) or direct current (DC).

The electrosurgical devices may further comprise a means for controllably applying the treatment surface of the semiconductor chip to the target site. Such means includes, for example, a handle directly or indirectly attached to the package. In some embodiments, the handle attaches to the second side of the package as described above.

The semiconductor chips according to the disclosure comprise at least one RF generator, and may include a plurality of RF generators. In particular embodiments, the semiconductor chips may include 2, 3, 4, 5, 6, or more RF generators, and in some instances, may include 12, 24, or more RF generators. It will be appreciated that, when more than one RF generator is present in a single device, such RF generators may be located on separate semiconductor chips or on a single semiconductor chip.

The RF generators that may be used in the devices of the invention are any suitable for incorporation onto a semiconductor chip and suitable for providing RF energy to a tissue treatment device. In preferred embodiments, a class-D RF generator is disposed on the semiconductor chip. Other types of RF may also be used, for example class-A or -AB generators, as will be appreciated by the skilled artisan.

In preferred embodiments, the RF generators are components located on the semiconductor die, and create one or more RF output signals. In general, the RF generators of the disclosure take a DC input signal and provide and RF output signal. The RF output signal may be sufficiently powerful for directly supplying the electrodes present in the devices of the disclosure. Alternatively, the RF signals provided by the RF generators may require amplification prior to reaching the electrodes. Such amplification may be obtained by an amplifier that is separate from the semiconductor chip upon which the RF generator is disposed, but in preferred embodiments, the semiconductor chip provides RF power sufficient to obviate the need for further amplification. The RF signal output of the semiconductor chips according to the invention may be in the range of 0.001 W to about 100 W, or within the range of about 0.01 W to about 40 W. In preferred embodiments, the output is at least 0.1 W, or at least 0.5 W, or at least 1 W, or at least 2 W, or at least 5 W, or at least 10 W, or at least 20 W. When the output of the semiconductor chip is amplified by an amplifier located internal or external to the semiconductor chip prior to reaching the electrodes, the output of the amplifier will also fall within these power values.

It will be appreciated, therefore, that the output signals of the RF generators disclosed herein may be modified by other components located on and/or off of the semiconductor chip. For example, the devices of the disclosure may include circuitry suitable for rectifying, amplifying, filtering, transforming, pulsing, attenuating, or otherwise modifying the output from the RF generators. In some embodiments, such circuitry is located on the semiconductor chip. In other embodiments, such circuitry is located external to the chip, for example on an adaptor such as a printed circuit board (PCB) adaptor.

For example, when a class-D generator is used as the RF generator, the square voltage waveform output may necessitate additional circuitry in the treatment device to convert the generator's output signal to the desired sinusoidal RF signal. Such circuitry may be located on the semiconductor chip (in addition to the RF generator), or may be located on an adaptor as described herein.

In embodiments that include a PCB adaptor, the semiconductor chip comprising one or more RF generators may be a component on the PCB adaptor, or may be separate from (but interfaced to) the adaptor. For devices comprising a plurality of RF generators located on a plurality of semiconductor chips, each chip may be disposed on the PCB adaptor. In preferred embodiments, the PCB adaptor comprises means for connecting the semiconductor chip to the electrodes, or to wires that connect to the electrodes.

In some embodiments, the semiconductor chip(s) and, when present, the adaptor and any connecting wires are contained within a treatment housing (also referred to herein as a “treatment probe”). The treatment housing preferentially will have a treatment surface, upon which one or more electrodes are disposed. The electrodes are electrically connected to the semiconductor chip and adaptor (when present), and are suitable for applying RF energy to the target tissue. In some preferred embodiments, the treatment housing will also contain a power source such as a battery.

Application of RF energy to the electrical contacts on the treatment surface of the package (or to electrodes disposed on the treatment housing) causes an electric field to be generated in the vicinity of the treatment surface. By placing the contacts in close proximity with a target site such as tissue, this electric field can be used to treat the tissue, as described herein. The electric field may be used in this way to induce electron movement within the tissue. Alternatively, the electrical contacts can be brought into direct contact with the tissue, thereby directly providing an electrical current within the tissue.

The electrosurgical semiconductor chip devices as described herein may be intended for single-use applications. In this case, the chips are disposable. Alternatively, the chips may be intended for multiple-use applications. In such cases, the chips may be capable of being appropriately cleaned between uses. Such methods of cleaning include washing with water or an appropriate solvent, and sterilization. In some embodiments, the semiconductor chips (i.e., the semiconductor dies and packages) are housed within an enclosure or disposed upon a support structure, and are electrically coupled to electrodes on a surface of a treatment housing. In such embodiments, the electrodes and the treatment housing in general are disposable or, alternatively, capable of being washed and/or sterilized.

As described herein, the electrosurgical devices comprise an electrosurgical semiconductor chip electrically coupled to a power source. The power source is preferentially a battery pack housed within the treatment housing, but may also be an external power source as is typically used for electrosurgical devices.

The electrosurgical devices described herein may employ RF energy as is commonly used for electrosurgical devices, or phase-controlled RF energy. Phase-controlled RF is described in co-pending U.S. application Ser. No. 11/654,914, the contents of which are incorporated by reference herein. In essence, in order to obtain phase-controlled RF energy, the electrodes are electrically coupled to a RF generator capable of providing a plurality of power outputs. The RF generator may comprise a plurality of RF sources, or may comprise a single RF source and appropriate circuitry to split the output of the RF source into a plurality of RF signals. The RF generator comprises (or is attached to) a means for controlling the phase between any two of the power outputs. Such means for controlling will typically consist of phase shifting circuitry and the like, as will be appreciated by one of ordinary skill in the art. The phase angle between at least two RF sources is adjustable, but it will be appreciated that the configuration of the electrosurgical devices may vary. In one embodiment, the RF generator comprises two RF sources and phase shifting circuitry for adjusting the phase angle between the RF outputs of the two RF sources. In another embodiment, the RF generator comprises first, second, and third RF sources. In one example of this embodiment, the phases of each RF source are adjustable, such that the phase angles between the first and second, second and third, and first and third RF sources may be independently varied. In another example of this embodiment, the first RF source has fixed output, and the phases of the second and third RF sources are adjustable. This configuration also allows adjustment of the phase angle between any two of the RF sources. In yet another example of this embodiment, the first and second RF sources have fixed output, and the phase of the third RF source is adjustable. This configuration allows adjustment of the phase angle between the first and third, and second and third RF sources. Adjustment of the phase angle between RF sources may be accomplished automatically via a feedback loop that maintains a fixed phase angle or responds to a measured electrical parameter (e.g., impendence at the target site, etc.), or may be accomplished manually via adjustment controls. It will be appreciated that the use of phase controlled RF energy allows: (a) treatment of the skin using lower voltages than would be necessary to achieve the same effect using non-phase-controlled RF energy; and/or (b) treatment of the skin to achieve medical effects that are not possible using non-phase-controlled RF energy.

It will also be appreciated that phase-controlled RF is only one method that may be used by the devices disclosed herein. Traditional RF energy (i.e., not phase-controlled) may also be applied to the treatment tissue.

The electrosurgical semiconductor chips disclosed herein employ a plurality of electrodes disposed on a treatment surface and adapted to be applied to a target biological tissue. The electrodes may be of any appropriate size or shape, and it will be appreciated that such will vary depending, for example, on the intended use. The treatment surface can be adapted to treat a variety of biological tissue surfaces. The electrodes may be uniformly disposed across the entire treatment surface, or may be concentrated in a particular section of the treatment surface. Typically, a regular pattern will be formed by the distribution of the electrodes on the treatment surface. The spacing between the electrode will depend, for example, on the semiconductor chip geometry and the size of the electrodes. Alternatively, in embodiments where the semiconductor chip is not intended to contact the target tissue (i.e., the chip is electrically connected to electrodes on a treatment surface of a treatment housing), the electrodes may be disposed on the treatment surface in any convenient manner. For treatment of human skin, for example, the center-to-center distance between adjacent electrodes may be between about 0.001 mm and about 100 mm, or between about 0.01 mm and about 25 mm. In one embodiment, adjacent electrodes are spaced apart an average of about 0.01 mm to about 1 mm.

As mentioned previously, the electrosurgical semiconductor chip may be disposable, such that it is sterilized upon manufacture and is intended for a one-time use. Alternatively, the electrosurgical semiconductor chip may be sterilizable (e.g., autoclavable) such that it is suitable for multiple uses and, in particular, use with multiple patients.

Alternatively, and as mentioned previously, the electrosurgical semiconductor chip is electrically connected to electrodes disposed on a treatment surface of a treatment probe. The treatment probe may have any convenient form, but will generally have a region suitable to be grasped and manipulated by the user of the device (e.g., a handle portion, or a gripping region on the probe) as well as the treatment surface.

In one embodiment, an electrosurgical device is provided that comprises a means for applying light energy to the treatment site. Such means for applying light energy include coherent sources and incoherent sources, and may include sources such as lasers, ultraviolet lamps, infrared lamps, incandescent and fluorescent lamps, light emitting diodes, and the like. The means for applying light may be attached to the electrosurgical semiconductor chip or may be separate from the electrosurgical semiconductor chip.

The electrosurgical device may comprise a means for measuring an electrical characteristic, and optionally a feedback loop that allows the electrosurgical device to adjust the supplied electrical energy in response to the measured electrical characteristic. Such electrical characteristics include the electrical impedance and/or admittance of the target site, the current flowing between electrodes, the electrical potential between electrodes, output voltages and phases of the RF sources, and phase differentials between RF sources. Such measurements may be taken in real time as the electrosurgical semiconductor chip is in close proximity to the target site, allowing the feedback loop to regulate the power supplied by the electrosurgical device to achieve the desired result.

Characteristics of the electrodes may be independently measured and monitored by appropriate circuitry. Furthermore, the RF power sources may be adapted to modify the electric field generated by the electrodes so as to reduce the current through one or more of the electrodes, substantially independently of the current through any of the other electrodes.

The electrosurgical devices described herein are useful in methods for delivering energy to a target site of a patient. Target sites suitable for the application of electrical energy using the devices disclosed herein include biological tissues such as skin, mucous membranes, organs, blood vessels, and the like. Energy is delivered to the target site via an electrosurgical semiconductor chip, which may be placed in close proximity to the target site. By “close proximity” is meant that the semiconductor chip is placed close enough to the target site to have a desired effect (e.g., tissue ablation, warming of the target site, etc.). In some embodiments, the electrosurgical semiconductor chip is placed in contact with the target site. In other embodiments, the semiconductor chip is housed within a treatment probe, and a treatment surface of the treatment probe is placed in close proximity to the target site.

In one embodiment, the target site is skin, and the electrosurgical device is placed in close proximity to the surface of the skin so as to generate an electric field that causes a current to flow through the stratum corneum, epidermis, and dermis. The induced electrical current may flow between electrodes, but may also have a significant component (e.g., 10%, 25%, 35%, 50%, 75% or more) in the direction that is perpendicular to the skin's surface. By creating an electrical current within the skin, the devices disclosed herein are able to increase the temperature of the skin, and in some cases, ablate one or more layers of skin. For example, the devices are useful in fully or partially ablating the surface of the skin. The devices are also useful in partially or fully ablating one or more layers below the surface of the skin.

In one embodiment, the electrosurgical devices may be used to non-homogeneously increase the temperature of biological tissue as described herein. In another embodiment, the electrosurgical devices may be used to increase the temperature of biological tissue within one or more regions that are narrow relative to either the size of the electrosurgical semiconductor chip or the size of the electrodes that are employed.

In one embodiment, the electrosurgical devices of the disclosure may be adapted to create one or more focal damage regions at the target site. Creation of such focal damage regions is also referred to herein as microablation. Focal damage regions are isolated regions within the target site wherein tissue necrosis occurs. The sizes, locations, number, relative arrangement, and other factors of the focal damage regions are determined by the physical and electrical parameters of the electrosurgical devices, as well as operating conditions of the devices when in operation. The creation of focal damage regions is facilitated by the use of phase-controlled RF. Additional details describing the creating and use of focal damage regions is provided in U.S. application Ser. No. 11/654,914. In preferred embodiments, microablation occurs in the epidermis of the treated tissue.

In some embodiments, the devices of the disclosure are capable of causing both microablation and deep tissue heating of the target tissue. By “deep tissue heating” is meant that the underlying layers of tissue are heated to a temperature greater than the overlying layers (e.g., surface layers) of tissue. For example, the dermis and/or stratum corneum may be heated to a greater extent than the epidermis, causing an increase in temperature of the internal layers of tissue that is greater than any increase in temperature of the surface layers of tissue.

It will be appreciated that the physical dimensions, density, total number, and distribution pattern of the focal damage regions may vary depending on the intended application. The number and arrangement of electrodes, the phase of the RF energy applied to the electrodes, and other factors are selected based on the desired therapeutic effect. It will also be appreciated that the typically small size of the electrodes present on the electrosurgical chips as disclosed herein allows highly selective treatment of the target site.

The devices of the disclosure may therefore be used to produce perpendicular heating (either ablative or non-ablative) of the tissue directly below the electrode(s) where the devices are applied to tissue. Such heating may produce fractional ablative skin rejuvenation, as described above (e.g., microablation), in tissue below the electrodes (when the electrodes are applied to the tissue). The devices may alternatively produce deep tissue heating below and between the region where the electrodes are applied to the tissue. The deep tissue heating may be achieved gradually via sustained application of RF energy, or more rapidly via shorter bursts of more intense RF energy (e.g., pulses). In some preferred embodiments, the devices of the disclosure produce both microablation and deep tissue heating. Such combination devices may create these effects simultaneously and in varying amounts, or the effects may be individually and selectively obtained by controlling the RF applied to the skin via the devices (e.g. using control circuitry, selector switches, etc.).

Microablation and/or deep tissue heating may, in some embodiments, be achieved using the devices disclosed herein operating at less than or equal to 50 W, or less than or equal to 30 W, or less than or equal to 25 W, or less than or equal to 15 W, or less than or equal to 10 W, or less than or equal to 5 W. Such power levels typically refer to the output of the RF generator disposed on the semiconductor chips disclosed herein, but are equally applicable to the power that is delivered to the electrodes (i.e., after any amplification, etc. that may be carried out by additional circuitry components as described herein).

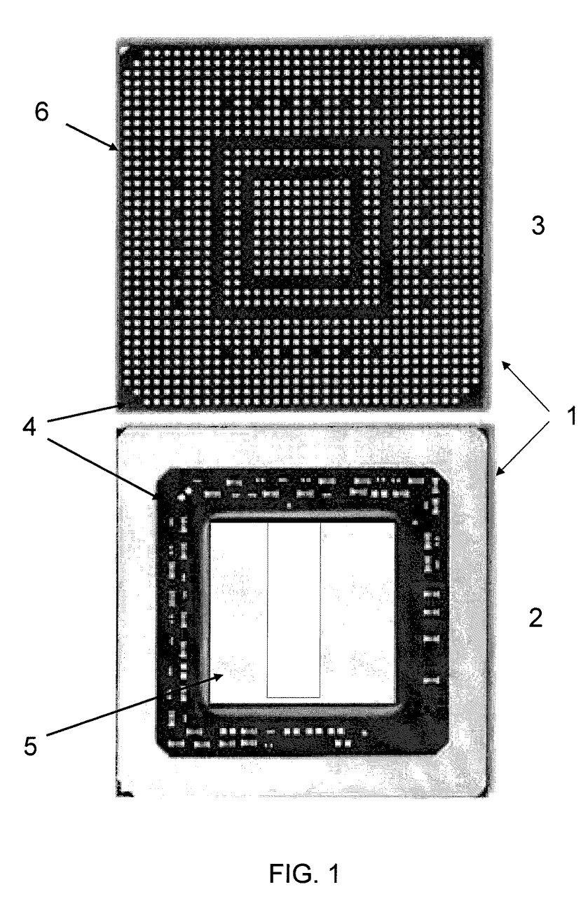

FIG. 1 shows top side 2 and bottom side 3 of electrosurgical chip device 1. Housed within package 4 is semiconductor chip 5. A plurality of ball electrodes 6 are disposed on the bottom side 3 of package 4.

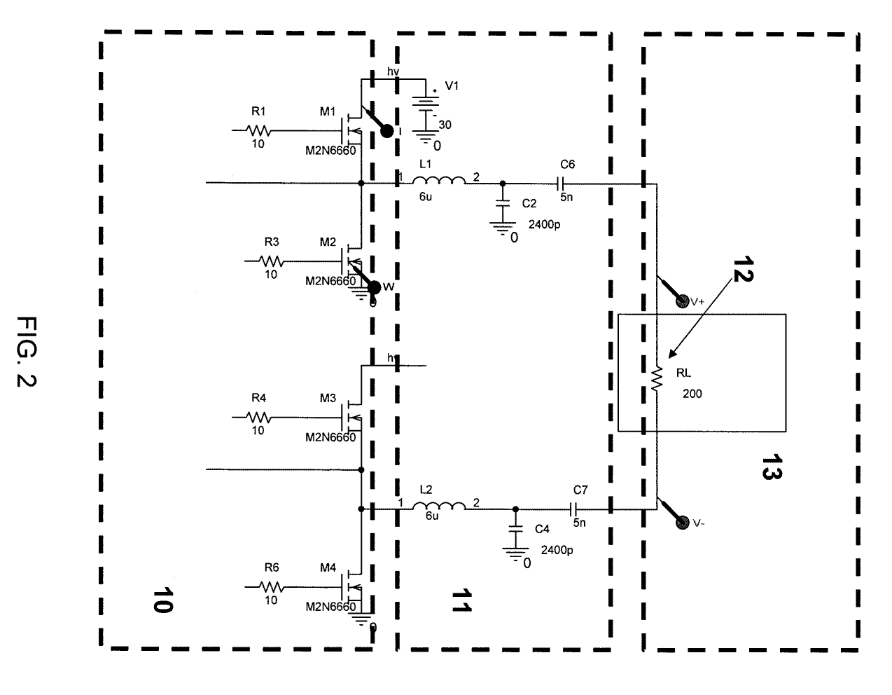

FIG. 2 shows chip output power stage 10, the RF output of which is passed through adaptor 11 prior to reaching electrode 12. Electrode 12 may be coupled to tissue region 13, thereby delivering RF energy to the tissue. The device provides, for example, RF power of about 10 W at 30 V input voltage when there is a resistance of 200 ohms.

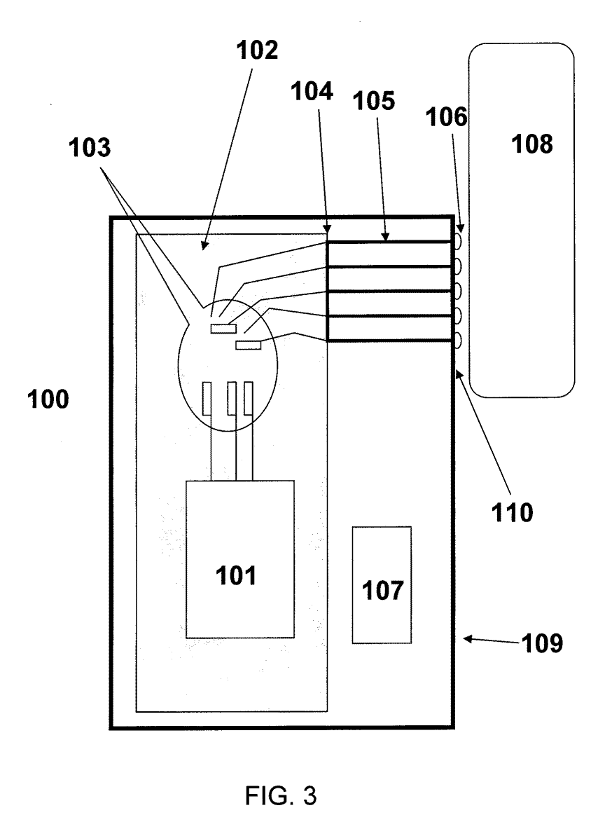

FIG. 3 shows, in block diagram format, the application of RF energy from electrosurgical chip device 100 to tissue 108. Semiconductor chip 101 is disposed on PCB adaptor 102. Also disposed on PCB adaptor 102 is complimentary circuitry 103, which may comprise filters, rectifiers, amplifiers, capacitors, inductors, resistors, and other components as described herein. PCB adaptor 102 is electrically connected to a plurality of electrodes 106 via connector 104 and wires 105. Semiconductor chip 101, PCB adaptor 102, connector 104, wires 105, and battery 107 are housed within treatment housing 109. Treatment housing 109 therefore provides a convenient package for the electrosurgical chip device 100, as well as a tissue treatment surface 110 with electrodes 106 disposed thereon. Electrosurgical chip device 100 may be completely self-contained (as shown in FIG. 3) or may contain connections to external devices (not shown) such as power supplies, power amplifiers, control units and the like.

The treatment surface of the electrosurgical device employing a semiconductor chip comprising one or more RF generators may be translated (i.e., moved) parallel to the skin surface during the application of electrical energy to the skin. Such translation may occur with the semiconductor chip either in contact with the skin or in close proximity to the skin. Translation of the semiconductor chip allows for enlarged areas of treatment, improved heat dissipation, and other benefits as will be appreciated by the skilled artisan. The RF sources can also be programmed and controlled, using standard control circuitry, to apply RF energy to the electrodes in a time-dependent fashion, such that specific patterns of focal damage regions are created based on the rate and direction of translation of the electrosurgical semiconductor chip.

In addition or as an alternative to creating focal damage regions, electrical energy applied via the electrosurgical devices disclosed herein may be used to heat, but not destroy and/or damage, the target site. For example, when the target site is skin, heat may be applied to affect collagen remodeling in a method for treating wrinkles.

The RF devices and methods as disclosed herein may be combined with other sources of energy. In some embodiments, the use of additional forms of energy allow synergistic effects for treatment of conditions such as skin disorders, skin aging and hair removal. For example, focused ultrasound energy may cause micro-vibrations in susceptible living tissue. The micro-vibrations caused by the ultrasound differ for different types of tissue (e.g., skin; keratinocytes or epidermal cells, hard keratin such as the shaft of hairs, etc.). Since focused ultrasound energy can differentiate physical properties of living tissue (e.g., treated from untreated tissue during electrosurgical procedures, adipose subdermal cells from connective tissue cells, etc.), it can amplify the selectivity of the effects of RF energy. In one embodiment of the methods and devices disclosed herein, RF (including phase-controlled RF) and ultrasound energy are used to treat tissue. Examples of uses for the combination of RF and ultrasound energy include the removal of hair and therapy of cellulite hair (e.g., hair removal or therapy that is safer and more efficient than existing methods).

The methods disclosed herein may further comprise a pretreatment step such as: treatment with a topical anesthetic; cooling; and treatment with light energy. Topical anesthetics such as lidocain and the like may be applied as needed, such as 30-60 minutes prior to treatment with the electrosurgical device. Cooling of the target site as a pretreatment step may involve application of cooling agents such as gels, liquids, or gases. Examples include water and saline solutions, liquid nitrogen, carbon dioxide, air, and the like. Cooling may also involve electrical contact cooling. Typically, cooling of the target site is accomplished just prior to treatment with the electrosurgical semiconductor chip, and has the effect of reducing pain and unwanted heat damage to the tissue surrounding the target site.

After treatment of the target site with the electrosurgical devices described herein, certain post-treatment steps may also be taken. Such post-treatment steps include treatment with a topical anesthetic as described above, and cooling of the target site and surrounding tissue as described above.

The electrosurgical methods and devices disclosed herein may also be used in conjunction with an additional means for applying energy such as electromagnetic and/or ultrasound energy to the target site. Such additional means for applying energy may be located on the electrosurgical semiconductor chip, or they may be separate and self contained.

The methods and devices disclosed herein are useful in the field of electrosurgery in general, and more specifically in procedures that are suitable for treatment using RF energy. For example, the methods and devices disclosed herein may be employed in procedures useful in the treatment of medical and aesthetic disorders and conditions affecting a patient's skin and subcutaneous tissue, including the following: skin resurfacing procedures; lessening the appearance of or removal of pigmentations; treating sun damaged and/or aged skin; lessening the appearance, removing, or otherwise treating cellulite; therapy or removal of wrinkles, vascular lesions, scars and tattoos; hair removal and hair transplant procedures; treatment of skin cancer; skin rejuvenation; treatment of acne and psoriasis; debridment of chronic skin ulcers; and blepharoplasty procedures.

The methods and devices disclosed herein are also useful in treating the signs of skin aging, including treatment of skin roughness, uneven pigmentation, wrinkles, and dilated capillaries.

Other applications for the devices and methods disclosed herein include removal of aging or diseased skin, thereby allowing fast regeneration by the non-ablated skin of the surrounding areas. The devices disclosed herein are also useful in methods for treating wrinkles and other signs of aging. Warming the collagen below the surface of the skin causes the collagen molecules to reorient on a molecular level, thereby eliminating or reducing the presence of wrinkles.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments thereof, that the foregoing description as well as the examples that follow, are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains.

### EXAMPLES

Three processes were evaluated in simulations. Three full design environments (PDK's) accordingly installed: (1) TSMC; (2) Atmel; and (3) MHS. PDK's are fully supporting Schematic, Layout, Analog and Digital cadence based simulation and verification design flow. The following issues were covered: Output Power and Power efficiencies. The power parameters are extracted at the following test conditions: (a) Output stage+Pure resistance load (200 Ohm); (b) Output stage+RLC circuit load; (c) Output stage+Implemented level shifter & pre-driver+RLC circuit.

Results indicate that: (1) High power (4 W-10 W) Ron driver stage can be implemented in all 3 processes, that is verified in respect to both Block level driver circuitry and Top level power/ground supply connectivity (IR drop and Current density induced electro migration); and (2) Output power efficiencies vary from 30% to 70% and 80% to 90% with processes. The lower range stand for real load and complete driver while the higher range stand for 200 Ohm load and output driver stage only.

What is claimed is:

1. A device for treating tissue comprising: a semiconductor die having at least one RF generator disposed thereon; a package housing the semiconductor die; and a plurality of electrodes electrically connected to the at least one RF generator.

2. The device of claim 1, wherein the semiconductor die and the package are housed within a treatment probe, and wherein the plurality of electrodes are disposed on a treatment surface of the treatment probe

3. The device of claim 2, wherein the semiconductor die and the package are disposed on a printed circuit board (PCB) adaptor.

4. The device of claim 1, wherein the semiconductor die comprises two or more RF generators, and wherein the device is configured to provide phase-controlled RF energy to the tissue.

5. The device of claim 1, wherein the package has a treatment surface, and wherein the plurality of electrodes are disposed on the treatment surface of the package.

6. The device of claim 5, wherein the package is mounted on a tissue treatment probe such that the electrodes can be brought into close proximity with the tissue.

7. The device of claim 1, wherein the plurality of electrodes are configured such that, upon the application of RF energy to the electrodes, an electric field is created suitable for delivering a therapeutic amount of RF energy to the tissue.

8. The device of claim 1, wherein the device is suitable for resurfacing skin, removing pigmentation, hair, wrinkles, scars, tattoos, or lesions from skin, treating sun-damaged skin, treating aged skin, rejuvenating skin, treating cellulite, treating acne, psoriasis, or cancer, debriding chronic skin ulcers, hair transplant procedures, or blepharoplasty procedures.

9. The device of claim 1, wherein the at least one RF generator provides RF energy to the plurality of electrodes in an amount that is sufficient to modify the tissue.

10. The device of claim 1, wherein the device is suitable for causing a tissue effect selected from microablation, deep tissue heating, and the combination thereof.

11. The device of claim 1, wherein the at least one RF generator is capable of providing at least one RF output signal; and wherein the device further comprises an adaptor for modifying the at least one RF output signal to create at least one modified RF signal.

12. The device of claim 4, wherein the phase-controlled RF energy is effective to cause ablation of at least a portion of the surface of the treatment tissue.

13. The device of claim 4, wherein the phase controlled RF energy is effective to cause non-homogeneous heating of the treatment tissue such that the increase in temperature of a region below the surface of the treatment tissue is greater than any increase in temperature of the surface of the treatment tissue.

14. A method for applying RF energy to tissue comprising contacting the tissue with one or more electrodes electrically coupled to an RF generator, the RF generator being disposed on a semiconductor chip.

15. The method of claim 14, wherein the one or more electrodes are disposed on a treatment surface of a treatment probe.

16. The method of claim 15, wherein the treatment probe houses the semiconductor chip.

17. The method of claim 15, wherein the semiconductor chip is housed within a control unit, and wherein the control unit is electrically coupled to the treatment probe.

18. The method of claim 15, wherein the RF device further comprises an adaptor connected between the semiconductor chip and the electrodes, wherein the adaptor is suitable for modifying the output of the semiconductor chip.

19. The method of claim 14, wherein the RF energy ablates at least a portion of the surface of the tissue such that the RF energy creates a plurality of microablation columns in the tissue, wherein the microablation channels comprise ablated tissue at the surface of the tissue.

20. The method of claim 14, wherein the tissue comprises surface tissue and underlying tissue, and wherein the RF energy heats the underlying tissue selectively over the surface tissue.
